(19) United States
(12) Patent Application Publication
(10) Pub. No.: US 2006/0078523 A1
(43) Pub. Date: Apr. 13, 2006

(54) Composition comprising at least one electrophilic monomer and at least one acid in a cosmetically acceptable anhydrous medium, and use thereof for cosmetic treatment of the hair

(76) Inventors: Gabin Vic, Venette (FR); Luc Gourlaouen, Asnieres (FR); Aude Livoreil, Paris (FR); Gaelle Brun, Paris (FR); Franck Giroud, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/248,335

(22) Filed: Oct. 13, 2005

(30) Foreign Application Priority Data
Oct. 13, 2004 (FR) 04 10813

(51) Int. Cl. A61K 8/81

(52) U.S. Cl. 424/70.11; 424/70.16

(57) ABSTRACT

Disclosed herein is a cosmetic composition comprising at least one electrophilic monomer and at least one non-reducing organic acid containing from 1 to 12 carbon atoms in a cosmetically acceptable anhydrous medium, and its use for the cosmetic treatment of the hair. Also disclosed herein is a method of cosmetic treatment of the hair which employs the composition.

The present application claims benefit under 35 U.S.C. § 119 to French Application No. 04 10813, filed Oct. 13, 2004, the contents of which are incorporated herein by reference.

Disclosed herein is a cosmetic composition comprising at least one electrophilic monomer and at least one acid, in a cosmetically acceptable anhydrous medium.

Also disclosed herein is the use of such a composition for the cosmetic treatment of hair, and a cosmetic treatment process using such a composition.

Within the field of cosmetology, attempts are made to modify the surface properties of the hair, for example to provide the hair with a conditioning effect, such as softness or sheen. This may be done using cosmetic compositions based on conditioning agents such as silicones or polymers which may have a high affinity for the hair.

However, these conditioning agents may be lost in the course of washing with shampoos, making it necessary to repeat application of the compositions to the hair.

In order to increase the staying power of polymer deposits, it may be possible to carry out a free-radical polymerization of certain monomers directly on the hair. However, severe degradation of the hair fibers may be observed, possibly associated with the polymerization initiators, and the hair thus treated may be difficult to disentangle.

It has been found that by combining a particular anhydrous cosmetically acceptable medium and at least one particular acid with at least one electrophilic monomer as described below it may be possible to obtain conditioning and sheen of the hair having improved durability.

One possible explanation for this improved durability is because a composition comprising a combination of ingredients as described herein may make it possible to maintain the softness and sheen provided to the hair by the composition, without repeat application, even after the hair has been washed a number of times.

Applying a composition comprising such a combination may lead to the formation in situ of a glossy, lubricating coating having staying power, for example after shampooing.

Moreover, the individual hairs may remain separated and may be styled without problem.

Accordingly, embodiments disclosed herein provide a cosmetic composition comprising at least one electrophilic monomer and at least one non-reducing organic acid containing from 1 to 12 carbon atoms, in a cosmetically acceptable anhydrous medium chosen from at least one of organic oils, silicones, mineral oils, plant oils, waxes, C₅-C₁₀ alkanes, acetone, methyl ethyl ketone, C₁-C₂₀ acid esters of C₁-C₈ alcohols, dimethoxyethane, diethoxyethane, C₁₀-C₃₀ fatty alcohols, C₁₀-C₃₀ fatty acids, C₁₀-C₃₀ fatty amides, and esters of C₁₀-C₃₀ fatty alcohols.

Embodiments disclosed herein may further provide a method of cosmetic treatment of the hair using the composition.

Other subjects, features, aspects, and advantages of the embodiments disclosed herein will appear more clearly throughout the description and examples that follow.

According to one embodiment disclosed herein, the composition comprises in a cosmetically acceptable anhydrous medium at least one electrophilic monomer and at least one non-reducing organic acid containing from 1 to 12 carbon atoms.

As used herein, the term “non-reducing” means a compound whose standard redox potential at 25° C. (relative to the hydrogen electrode) is greater than 0 volt.

As used herein, the term “cosmetically acceptable medium” means a medium that is compatible with the hair.

As used herein, the term “anhydrous medium” means a medium containing less than 1% by weight of water relative to the total weight of the composition.

The cosmetically acceptable medium may be chosen from at least one of organic oils; silicones, such as volatile silicones, amino silicone gums and oils, and non-amino silicone gums and oils; mineral oils; plant oils, such as olive oil, castor oil, rapeseed oil, coconut oil, wheatgerm oil, sweet almond oil, avocado oil, macadamia oil, apricot oil, safflower oil, candlenut oil, camelina oil, tamanu oil, and lemon oil; waxes; and organic compounds such as C₅-C₁₀ alkanes, acetone, methyl ethyl ketone, C₁-C₂₀ acid esters of C₁-C₈ alcohols, such as methyl acetate, butyl acetate, ethyl acetate, and isopropyl myristate, dimethoxyethane, diethoxyethane, C₁₀-C₃₀ fatty alcohols, such as lauryl alcohol, cetyl alcohol, stearyl alcohol, and behenyl alcohol, C₁₀-C₃₀ fatty acids, such as lauric acid and stearic acid, C₁₀-C₃₀ fatty amides, such as lauric diethanolamide, and esters of C₁₀-C₃₀ fatty alcohols, such as C₁₀-C₃₀ fatty alkyl benzoates.

In certain embodiments, the organic compounds comprising the cosmetically acceptable medium may be chosen from compounds that are liquid at a temperature of 25° C. and at 10⁵ Pa (760 mmHg).

In certain embodiments, the cosmetically acceptable medium may be chosen from at least one of olive oil, castor oil, rapeseed oil, coconut oil, wheatgerm oil, sweet almond oil, avocado oil, macadamia oil, apricot oil, safflower oil, candlenut oil, camelina oil, tamanu oil, lemon oil, polybutene oil, isononyl isononanoate, isostearyl malate, pentaerythrityl tetraisostearate, tridecyl trimellitate, and mixtures comprising cyclopentasiloxane (14.7% by weight) and α, ω-dihydroxylated polydimethylsiloxane (85.3% by weight).

The at least one non-reducing organic acid that may be used according to certain embodiments may be chosen from carboxylic acids, sulfonic acids, phosphonic acids, phosphinic acids, phosphoric monoesters, and phosphoric diesters. The organic acids mentioned all contain from 1 to 12 carbon atoms.

The at least one non-reducing organic acid that may be used according to certain embodiments disclosed herein may have a pKa ranging from 0 to 6.

Examples of at least one non-reducing organic acid that may be mentioned include acetic acid, formic acid, propionic acid, butyric acid, benzoic acid, mono-, di-, and trichloroacetic acid, salicylic acid, trifluoroacetic acid, benzenesulfonic acid, toluenesulfonic acid, methylphosphonic acid, methylphosphinic acid, dimethylphosphinic acid, and phosphonic acid monobutyl ester.

In certain embodiments, the at least one non-reducing organic acid does not comprise any PO₄ subunits.

A non-reducing organic acid that may be used according to certain embodiments is acetic acid.

The at least one non-reducing organic acid as described above may be present in the cosmetic compositions disclosed herein in an amount ranging from 0.001 % to 20% by weight, such as from 0.002% to 10% by weight or from 0.005% to 5% by weight, relative to the total weight of the composition.

As used herein, an electrophilic monomer is a monomer capable of undergoing polymerization by anionic polymerization in the presence of a nucleophile such as, for example, the hydroxyl (OH⁻) ions present in water.

As used herein, the term “anionic polymerization” means the mechanism defined in the work “Advanced Organic Chemistry”, Third Edition, Jerry March, pages 151 to 161.

The at least one electrophilic monomer present in the compositions disclosed herein may be chosen from:

(i) benzylidenemalononitrile derivatives (A), 2-(4-chloro-benzylidene)malononitrile (A1), ethyl 2-cyano-3-phenylacrylate (B), and ethyl 2-cyano-3-(4-chlorophenyl) acrylate (B1), as described in Sayyah, J. Polymer Research, 2000, p. 97:

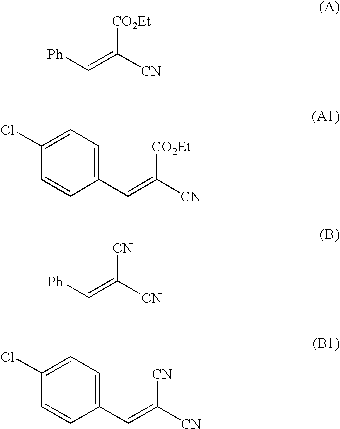

(ii) methylidenemalonate derivatives such as:

diethyl 2-methylenemalonate (C) as described by Hopff, Makromolekulare Chemie, 1961, p. 95; by De Keyser, J. Pharm. Sci, 1991, p. 67; and by Klemarczyk, Polymer, 1998, p.173:

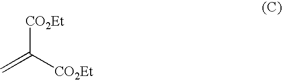

ethyl 2-ethoxycarbonylmethyleneoxycarbonylacrylate (D), as described by Breton, Biomaterials, 1998, p. 271 and Couvreur, Pharmaceutical Research, 1994, p. 1270:

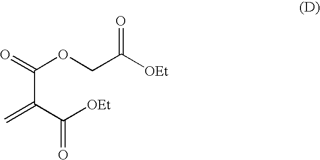

(iii) itaconate and itaconimide derivatives such as:

dimethyl itaconate (E), as described by Bachrach, European Polymer Journal, 1976, p. 563:

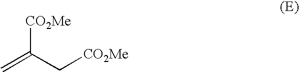

N-butylitaconimide (F), N-(4-tolyl)itaconimide (G), N-(2-ethyl-phenyl)itaconimide (H) and N-(2,6-diethylphenyl)itaconimide (I), as described by Wanatabe, J. Polymer Science: Part A: Polymer Chemistry, 1994, p.2073: 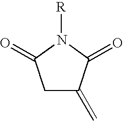
R=Bu (F), 4-tolyl (G), 2-ethylphenyl (H), 2,6-diethylphenyl (I)

(iv) methyl α-(methylsulphonyl)acrylate derivatives (K), ethyl α-(methylsulphonyl)acrylate derivatives (L), methyl α-(tert-butylsulphonyl)acrylate derivatives (M), tert-butyl α-(methylsulphonyl)acrylate derivatives (N), and tert-butyl α-(tert-butylsulphonyl)acrylate derivatives (O), as described by Gipstein, J. Org. Chem, 1980, p. 1486, and 1,1-bis(methylsulphonyl)ethylene derivatives (P), 1-acetyl-1-methylsulphonylethylene derivatives (Q), methyl α-(methylsulphonyl)vinylsulphonate derivatives (R), and α-methylsulphonylacrylonitrile derivatives (S), as described in U.S. Pat. No.2,748,050 to Shearer, et al.:

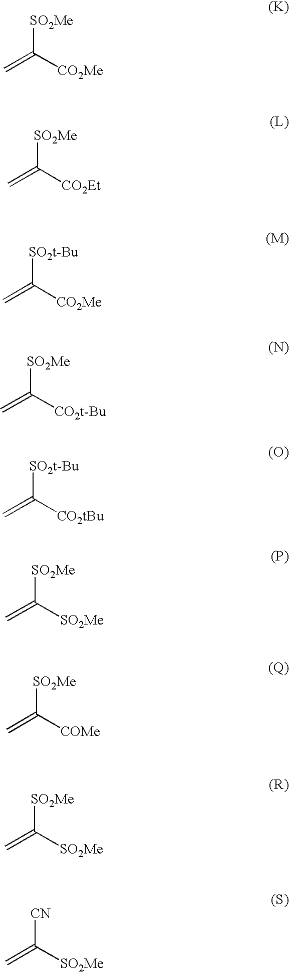

(v) methyl vinyl sulphone derivatives (T) and phenyl vinyl sulphone derivatives (U), as described by Boor, J. Polymer Science, 1971, p. 249:

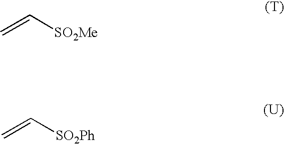

(vi) the phenyl vinyl sulphoxide derivative (V), as described by Kanga, Polymer Preprints (ACS, Division of Polymer Chemistry), 1987, p. 322:

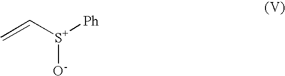

(vii) the 3-methyl-N-(phenylsulphonyl)-1-aza-1,3-butadiene derivative (W), as described by Bonner, Polymer Bulletin, 1992, p. 517:

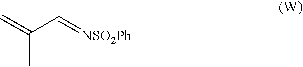

(viii) acrylate and acrylamide derivatives such as:

N-propyl-N-(3-triisopropoxysilylpropyl)acrylamide (X) and N-propyl-N-(3-triethoxysilylpropyl)acrylamide (Y), as described by Kobayashi, Journal of Polymer Science, Part A: Polymer Chemistry, 2005, p. 2754:

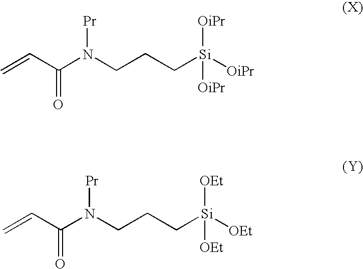

2-hydroxyethyl acrylate (Z) and 2-hydroxyethyl methacrylate (AA), as described by Rozenberg, International Journal of Plastics Technology, 2003, p.17:

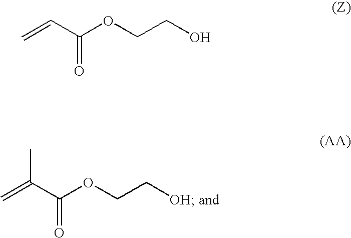

n-butyl acrylate (AB), as described by Schmitt, Macromolecules, 2001, p. 2115, and tert-butyl acrylate (AC), as described by Ishizone, Macromolecules, 1999, p. 955:

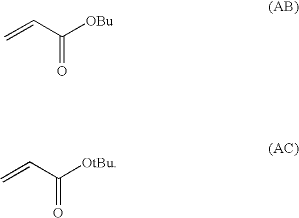

The at least one electrophilic, or electron-withdrawing, monomer disclosed herein may be cyclic or linear. When it is cyclic, the electron-withdrawing group may be exocyclic, which is to say that it does not form an integral part of the cyclic structure of the monomer.

According to one embodiment, these monomers may have at least two electron-withdrawing groups.

Examples of electrophilic monomers having at least two electron-withdrawing groups include the monomers of formula (I): 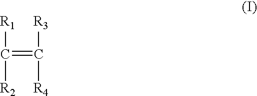
in which:

R₁ and R₂ are each chosen from, independently of one another, minimally- or non- electron-withdrawing groups (with little or no inductive withdrawal effect), such as:

hydrogen,

saturated or unsaturated linear, branched, or cyclic hydrocarbon groups, for example groups containing 1 to 20, such as 1 to 10, carbon atoms, and optionally comprising at least one atom chosen from nitrogen, oxygen, and sulphur atoms, optionally substituted by at least one group chosen from from —OR, —COOR, —COR, —SH, —SR, —OH, and halogen atoms,

modified or non-modified polyorganosiloxane residues, and

polyoxyalkylene groups;

R₃ and R₄ are each chosen from, independently of one another, electron-withdrawing (or inductively withdrawing) groups chosen from the groups —N(R)₃⁺, —S(R)₂⁺, —SH₂⁺, —NH₃⁺, —NO₂, —SO₂R, —C═N, —COOH, —COOR, —COSR, —CONH₂, —CONHR, —F, —Cl, —Br, —I, —OR, —COR, —SH, —SR, —OH, linear or branched alkenyl groups, linear or branched alkynyl groups, C₁-C₄ monofluoroalkyl groups, C₁-C₄ polyfluoroalkyl groups, aryl groups such as phenyl, and aryloxy groups such as phenyloxy; and

R is chosen from saturated or unsaturated linear, branched, or cyclic hydro-carbon groups, for example groups containing 1 to 20, such as 1 to 10, carbon atoms, and optionally comprising at least one atom chosen from nitrogen, oxygen, and sulphur atoms, optionally substituted by at least one group chosen from —OR′, —COOR′, —COR′, —SH, —SR′, —OH, halogen atoms, and polymer residues obtainable by free-radical polymerization, by polycondensation, or by ring opening, wherein R′ is chosen from C₁-C₁₀ alkyl groups.

As used herein, electron-withdrawing or inductively withdrawing groups mean any group which is more electronegative than carbon. Reference may be made to the work by P. R. Wells in Prog. Phys. Org. Chem., Vol. 6, 111 (1968).

As used herein, a minimally or non-electron-withdrawing group is any group whose electronegativity is less than or equal to that of carbon.

The alkenyl and alkynyl groups disclosed herein may have 2 to 20 carbon atoms, for example 2 to 10 carbon atoms.

As a saturated or unsaturated linear, branched, or cyclic hydrocarbon group, containing, for example, 1 to 20 carbon atoms, such as 1 to 10 carbon atoms, mention may be made of linear or branched alkyl, alkenyl, and alkynyl groups, such as methyl, ethyl, n-butyl, tert-butyl, isobutyl, pentyl, hexyl, octyl, butenyl, and butynyl groups; cycloalkyl groups; and aromatic groups.

As a substituted hydrocarbon group, mention may be made, for example, of hydroxyalkyl groups and polyhaloalkyl groups.

As examples of non-modified polyorganosiloxanes, mention may be made of polyalkylsiloxanes such as polydimethylsiloxanes, polyarylsiloxanes such as polyphenylsiloxanes, and polyarylalkylsiloxanes such as polymethylphenylsiloxanes.

Among modified polyorganosiloxanes, mention may be made of polydimethylsiloxanes containing at least one group chosen from polyoxyalkylene, siloxy, silanol, amine, imine, and fluoroalkyl groups.

Among polyoxyalkylene groups, mention may be made of polyoxyethylene groups and polyoxypropylene groups having, for example, 1 to 200 oxyalkylene units.

Among monofluoroalkyl and polyfluoroalkyl groups, mention may be made of groups such as —(CH₂)ₙ—(CF₂)ₘ—CF₃ and —(CH₂)ₙ—(CF₂)ₘ—CHF₂ wherein n ranges from 1 to 20 and m ranges from 1 to 20.

The substituents R₁ to R₄ may optionally be substituted by a group which has a cosmetic activity. The cosmetic activities used may, for example, be obtained from groups having coloring, antioxidant, UV filter, and conditioning functions.

Examples of groups having a coloring function include azo, quinone, methine, cyanomethine, and triarylmethane groups.

Examples of groups having an antioxidant function include butylated hydroxyanisole (BHA) groups, butylated hydroxytoluene (BHT) groups, and vitamin E.

Examples of groups having a UV filter function include benzophenone, cinnamate, benzoate, benzylidenecamphor, and dibenzoylmethane groups.

Examples of groups having a conditioning function include cationic groups and fatty ester groups.

Among the above-mentioned monomers, mention may be made of the monomers from the class of the cyanoacrylates and their derivatives, of formula (II):

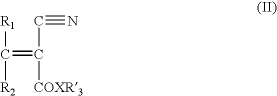

where

X is chosen from NH, S, and O;

R₁ and R₂ are as defined above; and

R′₃ is chosen from hydrogen and groups R as defined in the formula (I).

In certain embodiments, X denotes O.

As compounds of formula (II), mention may be made of the monomers:

a) belonging to the class of C₁-₂₀ perfluoroalkyl 2-cyanoacrylates, such as: the 2,2,3,3-tetrafluoropropyl ester of 2-cyano-2-propenoic acid, of formula: 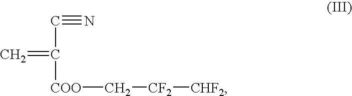
and the 2,2,2-trifluoroethyl ester of 2-cyano-2-propenoic acid, of formula:

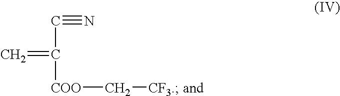

b) C₁-C₁₀ alkyl and (C₁-C₄)alkoxy(C₁-C₁₀)alkyl cyanoacrylates.

Mention may also be made of ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyano-acrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, and isoamyl cyanoacrylate.

According to certain embodiments, monomers b) may be used.

In certain embodiments, the at least one electrophilic monomer is chosen from those of formula V and mixtures thereof: 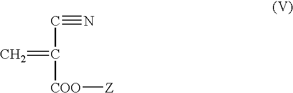
in which: Z is chosen from —(CH₂)₇—CH₃; —CH(CH₃)—(CH₂)₅—CH₃; —CH₂—CH(C₂H₅)—(CH₂)₃—CH₃; —(CH₂)₅—CH(CH₃)—CH₃; and —(CH₂)₄—CH(C₂H₅)—CH₃.

The at least one electrophilic monomer used in accordance with certain embodiments disclosed herein may be attached covalently to supports such as polymers, oligomers, and dendrimers. The polymer or oligomer may be of linear, branched, comb, or block structure. The distribution of the at least one electrophilic monomer disclosed herein over the polymeric, oligomeric, and dendritic structure may be random, terminal, or blockwise.

According to certain embodiments disclosed herein, the at least one electrophilic monomer may be chosen from monomers capable of undergoing polymerization on keratin fibers under cosmetically acceptable conditions. For example, the polymerization of the at least one electrophilic monomer may take place at a temperature less than or equal to 80° C., such as a temperature ranging from 10 to 80° C., or from 20 to 80° C., wherein the temperature does not prevent the application being ended by drying under a hood, blow-drying, or passage of a flat iron or curling tongs.

The compositions used in accordance with the embodiments disclosed herein may have a concentration of electrophilic monomer ranging from 0.001% by 80% by weight, such as ranging from 0.002% to 75% by weight, from 0.1% to 40% by weight, or from 1% to 20% by weight, relative to the total weight of the composition.

It is also possible to introduce at least one polymerization inhibitor into the compositions disclosed herein, such as free-radical polymerization inhibitors and anionic polymerization inhibitors, in order to increase the stability of the composition over time. Without limitation, the following polymerization inhibitors may be mentioned: sulphur dioxide; nitric oxide; lactone; boron trifluoride; hydroquinone and its derivatives such as hydroquinone monoethyl ether and tert-butylhydroquinone (TBHQ); benzoquinone and its derivatives such as duroquinone; catechol and its derivatives such as tert-butylcatechol and methoxycatechol; anisole and its derivatives such as methoxyanisole, hydroxyanisole, and butylated hydroxyan isole; pyrogallol; 2,4-dinitrophenol; 2,4,6-trihydroxybenzene; p-methoxyphenol; hydroxybutyltoluene; alkyl sulphates; alkyl sulphites; alkyl sulphones; alkyl sulphoxides; alkyl sulphides; mercaptans; and 3-sulpholene. The alkyl groups may, for example, be chosen from groups having 1 to 6 carbon atoms.

The amount of the at least one polymerization inhibitor may range from 10 ppm to 20%, such as from 10 ppm to 5% or from 10 ppm to 1%, by weight relative to the total weight of the composition.

The compositions disclosed herein may further comprise at least one agent which is commonly used in cosmetology, chosen, for example, from reducing agents; fats; plasticizers; softeners; antifoams; moisturizers; pigments; clays; mineral fillers; UV filters; mineral colloids; peptizers; solubilizers; perfumes; preservatives; anionic, cationic, nonionic, or amphoteric surfactants; fixative polymers; non-fixative polymers; polyols; proteins; vitamins; direct dyes; oxidation dyes; pearlizers; propellants; and organic or inorganic thickeners such as benzylidenesorbitol and N-acylamino acids.

These agents may be optionally encapsulated. The capsule may be a polycyanoacrylate capsule.

The compositions disclosed herein may be used on the hair, optionally in the presence of at least one nucleophile, for the cosmetic treatment thereof.

A method of cosmetic treatment according to certain embodiments disclosed herein comprises applying a composition as defined above to the hair, in the presence of at least one nucleophile as defined below.

Nucleophiles capable of initiating the anionic polymerization are systems known per se which are capable of generating a carbanion on contact with an electrophilic monomer. As used herein, “carbanion” means the chemical species defined in “Advanced Organic Chemistry”, Third Edition, Jerry March, page 141.

The at least one nucleophile disclosed herein may be composed of a molecular compound, an oligomer, a dendrimer, or a polymer which possesses nucleophilic functions. Without limitation, nucleophilic functions which may be mentioned include the following functions: R₂N⁻, NH₂⁻, Ph₃C⁻, R₃C⁻, PhNH⁻, pyridine, ArS⁻, R—C═C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻, and H₂O, where Ph represents a phenyl group, Ar represents an aryl group, and R is chosen from C₁-C₁₀ alkyl groups.

In certain embodiments, the at least one nucleophile is chosen from hydroxyl ions, such as those present in water. This water may be provided by prior wetting of the hair.

In other embodiments, the composition comprising at least one electrophilic monomer does not contain any nucleophile.

In still other embodiments, the at least one nucleophile is contained in a second composition, used just before or after the composition comprising the at least one electrophilic monomer.

It is also possible, in order to modify the reaction kinetics, to wet the hair beforehand by means of an aqueous solution whose pH has been adjusted using a base, an acid, or an acid/base mixture. The acid and/or the base may be organic or inorganic.

It is also possible to modify the anionic polymerization kinetics by pre-impregnating the hair, using at least one nucleophile other than water. The at least one nucleophile may be used pure, in solution, in the form of an emulsion, or it may be encapsulated.

In order to modify the anionic polymerization kinetics, it is also possible to enhance the nucleophilicity of the hair by chemically converting the keratin material.

By way of example of chemical conversion, mention may be made of the reduction of the disulphide bridges, of which the keratin is partly composed, to thiols, prior to application of the composition disclosed herein. In a non-exhaustive listing, mention may be made, as reductants of the disulphide bridges of which the keratin is partly composed, of the following compounds:

anhydrous sodium thiosulphate,

powdered sodium metabisulphite,

thiourea,

ammonium sulphite,

thioglycolic acid,

thiolactic acid,

ammonium thiolactate,

glycerol monothioglycolate,

ammonium thioglycolate,

thioglycerol,

2,5-dihydroxybenzoic acid,

diammonium dithioglycolate,

strontium thioglycolate,

calcium thioglycolate,

zinc formaldehyde-sulphoxylate,

isooctyl thioglycolate,

dl-cysteine, and

monoethanolamine thioglycolate.

In order to modify the anionic polymerization kinetics, for example, to reduce the polymerization rate of the at least one electrophilic monomer disclosed herein, it is possible to enhance the viscosity of the composition. To accomplish this it is possible to add to the composition at least one polymer which exhibits no reactivity with the at least one electrophilic monomer. In this context, mention may be made, non-exhaustively, of poly(methyl methacrylate) (PMMA) and cyanoacrylate-based copolymers as described in U.S. Pat. No. 6,224,622.

In order to improve, among other things, the adhesion of the poly(cyanoacrylate) formed in situ, it is possible to pretreat the hair with polymers, or to carry out a hair treatment before applying the composition disclosed herein, such as a direct dyeing treatment, an oxidation dyeing treatment, a permanent waving treatment, or a straightening treatment.

Application of the compositions as described above may be optionally followed by rinsing.

The compositions disclosed herein may be in the form of a lotion, spray, or foam and may be applied as a shampoo or conditioner.

Another embodiment disclosed herein comprises a kit comprising a first composition containing at least one electrophilic monomer as defined above and optionally comprising at least one free-radical and anionic polymerization inhibitor as defined above, and a second composition comprising at least one non-reducing organic acid containing from 1 to 12 carbon atoms as defined above in a cosmetically acceptable anhydrous medium chosen from at least one of organic oils, silicones, mineral oils, plant oils, waxes, C₅-C₁₀ alkanes, acetone, methyl ethyl ketone, C₁-C₂₀ acid esters of C₁-C₈ alcohols, dimethoxyethane, diethoxyethane, C₁₀-C₃₀ fatty alcohols, C₁₀-C₃₀ fatty acids, C₁₀-C₃₀ fatty amides, and esters of C₁₀-C₃₀ fatty alcohols.

Further disclosed herein is a method of cosmetic treatment of the hair, comprising at least two steps, one step comprising applying the at least one non-reducing organic acid containing from 1 to 12 carbon atoms as defined above, and a second step comprising applying the at least one electrophilic monomer as defined above, the order of the steps being arbitrary.

One embodiment disclosed herein comprises applying the at least one non-reducing organic acid before applying the at least one electrophilic monomer.

In the examples below, all amounts are indicated in percent by weight of active substance relative to the total weight of the composition, unless indicated otherwise. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements. The following examples are intended to illustrate the invention without limiting the scope as a result.

### EXAMPLES

### Example 1:

A sheen cream according to one embodiment disclosed herein (Example 1) and a comparative sheen cream (Comparative Example 1) were prepared from the following ingredients:

| | Ex. 1 | Comp. Ex. 1 |
|---|---|---|
| N-Octyl 2-cyanoacrylate | 5% | 5% |
| Cyclopentasiloxane | 47.35% | 47.4% |
| Cyclopentasiloxane dimethicone copolyol | 47.4% | 47.4% |
| Acetic acid | 0.05% | — |
| Fragrance | 0.2% | 0.2% |

Mode of Application

Locks of 2.7 g of moderately sensitized hair were moistened with 1 ml of water per lock. 2 g of each of the compositions of Example 1 and Comparative Example 1 were applied to these moistened locks. After application, the locks of hair were dried under a drying hood for 30 minutes at 40° C.

For each lock, the feel and the sheen of the hair were evaluated by a panel of 10 people. A virgin lock of the same nature was used as a reference.

The tactile and visual evaluation of the various locks of hair were repeated with the same procedure after washing the locks 5 times successively with shampoo sold under the name DOP camomile.

The sensory evaluation results are indicated in the following table:

| Nature of the treatment | Ex. 1 | Comp. Ex. 1 |
|---|---|---|
| After application | Softness 5 | Softness 3 |
| | Sheen 5 | Sheen 3 |
| After 5 shampoo washes | Softness 4 | Softness 2 |
| | Sheen 4 | Sheen 2 |

The grading is:
0=equivalent to the untreated lock
5=very much greater than the untreated lock.

The softness and sheen provided by one embodiment of the composition disclosed herein (Example 1) were greater than those provided by the composition of Comparative Example 1 immediately after applying the composition.

After 5 shampoo washes, the softness and sheen provided are better conserved when the locks are treated with the composition of the invention (Example 1).

### Examples 2 to 7

The compositions detailed below illustrate other examples of sheen creams in accordance with embodiments of the invention.

### Example 2

| | Mass percentage |
|---|---|
| Methylheptyl cyanoacrylate (1) | 10% |
| Cyclopentasiloxane | 44.55% |
| Cyclopentasiloxane dimethicone copolyol | 45% |
| Acetic acid | 0.25% |
| Fragrance | 0.2% |

(1) sold by the company Chemence

### Example 3

| | Mass percentage |
|---|---|
| Ethoxyethyl cyanoacrylate (1) | 10% |
| Cyclopentasiloxane | 43.8% |
| Cyclopentasiloxane dimethicone copolyol | 45% |
| Acetic acid | 1% |
| Fragrance | 0.2% |

(1) EO 460 sold by the company Tong Shen

### Example 4

| | Mass percentage |
|---|---|
| Butyl cyanoacrylate (1) | 10% |
| Cyclopentasiloxane | 43.8% |
| Cyclopentasiloxane dimethicone copolyol | 45% |
| Acetic acid | 1% |
| Fragrance | 0.2% |

(1) B 60 sold by the company Tong Shen

### Example 5

| | Mass percentage |
|---|---|
| Ethylhexyl cyanoacrylate (1) | 10% |
| Cyclopentasiloxane | 44.55% |
| Cyclopentasiloxane dimethicone copolyol | 45% |
| Acetic acid | 0.25% |
| Fragrance | 0.2% |

(1) O 60 sold by the company Tong Shen

### Example 6

| | Mass percentage |
|---|---|
| Methylheptyl cyanoacrylate (1) | 9% |
| Ethylhexyl cyanoacrylate (2) | 1% |
| Cyclopentasiloxane | 44.55% |
| Cyclopentasiloxane dimethicone copolyol | 45% |
| Acetic acid | 0.25% |
| Fragrance | 0.2% |

(1) sold by the company Chemence
(2) O 60 sold by the company Tong Shen

### Example 7

| | Mass percentage |
|---|---|
| Methylheptyl cyanoacrylate (1) | 7% |
| Butyl cyanoacrylate (2) | 3% |
| Cyclopentasiloxane | 44.55% |
| Cyclopentasiloxane dimethicone copolyol | 45% |
| Acetic acid | 0.25% |
| Fragrance | 0.2% |

(1) sold by the company Chemence
(2) B-60 sold by the company Tong Shen

What is claimed is:

1. A cosmetic composition comprising at least one electrophilic monomer and at least one non-reducing organic acid containing from 1 to 12 carbon atoms, in a cosmetically acceptable anhydrous medium chosen from at least one of organic oils, silicones, mineral oils, plant oils, waxes, C₅-C₁₀ alkanes, acetone, methyl ethyl ketone, C₁-C₂₀ acid esters of C₁-C₈ alcohols, dimethoxyethane, diethoxyethane, C₁₀-C₃₀ fatty alcohols, C₁₀-C₃₀ fatty acids, C₁₀-C₃₀ fatty amides, and esters of C₁₀-C₃₀ fatty alcohols.

2. The composition according to claim 1, wherein the at least one non-reducing organic acid is chosen from carboxylic acids, sulfonic acids, phosphonic acids, phosphinic acids, phosphoric monoesters, and phosphoric diesters.

3. The composition according to claim 1, wherein the at least one non-reducing organic acid is chosen from acetic acid, formic acid, propionic acid, butyric acid, benzoic acid, monochloroacetic acid, dichloroacetic acid, trichloroacetic acid, salicylic acid, trifluoroacetic acid, benzenesulfonic acid, toluenesulfonic acid, methylphosphonic acid, methylphosphinic acid, dimethylphosphinic acid, and phosphoric acid monobutyl ester.

4. The composition according to claim 3, wherein the at least one non-reducing organic acid is acetic acid.

5. The composition according to claim 1, wherein the at least one non-reducing organic acid is present in an amount ranging from 0.001% to 20% by weight, relative to the total weight of the composition.

6. The composition according to claim 5, wherein the at least one non-reducing organic acid is present in an amount ranging from 0.002% to 10% by weight, relative to the total weight of the composition.

7. The composition according to claim 1, wherein the at least one electrophilic monomer is chosen from the formula: 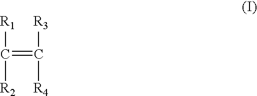
in which:
R₁ and R₂ are each chosen from, independently of one another, minimally or non-electron-withdrawing groups chosen from:
hydrogen,
saturated or unsaturated linear, branched, or cyclic hydrocarbon groups containing 1 to 20 carbon atoms and optionally comprising at least one atom chosen from nitrogen, oxygen, and sulphur atoms, optionally substituted by at least one group selected from —OR, —COOR, —COR, —SH, —SR, —OH, and halogen atoms,
modified or unmodified polyorganosiloxane residues, and
polyoxyalkylene groups;
R₃ and R₄ are each chosen from, independently of one another, electron-withdrawing groups chosen from the groups —N(R)₃⁺, —S(R)₂⁺, —SH₂⁺, —NH₃⁺, —NO₂, —SO₂R, —C═N, —COOH, —COOR, —COSR, —CONH₂, —CONHR, —F, —Cl, —Br, —I, —OR, —COR, —SH, —SR, —OH, linear or branched alkenyl groups, linear or branched alkynyl groups, C₁-C₄ monofluoroalkyl groups, C₁-C₄ polyfluoroalkyl groups, aryl groups, and aryloxy groups; and
R is chosen from saturated or unsaturated linear, branched, or cyclic hydrocarbon groups containing 1 to 20 carbon atoms and optionally comprising at least one atom chosen from nitrogen, oxygen, and sulphur atoms, optionally substituted by at least one group chosen from —OR′, —COOR′, —COR′, —SH, —SR′, —OH, halogen atoms, and polymer residues obtainable by free-radical polymerization, polycondensation, or ring opening, wherein R′ is chosen from C₁-C₁₀ alkyl groups.

8. The composition according to claim 7, wherein the at least one electrophilic monomer is chosen from the compounds of formula: 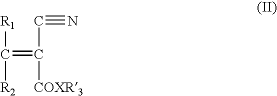
wherein
X is chosen from NH, S, and O,
R₁ and R₂ are each chosen from, independently of one another, minimally or non-electron-withdrawing groups chosen from:
hydrogen,
saturated or unsaturated linear, branched, or cyclic hydrocarbon groups containing 1 to 20 carbon atoms and optionally comprising at least one atom chosen from nitrogen, oxygen, and sulphur atoms, optionally substituted by at least one group selected from —OR, —COOR, —COR, —SH, —SR, —OH, and halogen atoms,
modified or unmodified polyorganosiloxane residues, and
polyoxyalkylene groups; and
R′₃ is chosen from hydrogen; and groups R,
wherein R is chosen from saturated or unsaturated linear, branched, or cyclic hydrocarbon groups containing 1 to 20 carbon atoms and optionally comprising at least one atom chosen from nitrogen, oxygen, and sulphur atoms, optionally substituted by at least one group chosen from —OR′, —COOR′, —COR′, —SH, —SR′, —OH, halogen atoms, and polymer residues obtainable by free-radical polymerization, polycondensation, or ring opening, wherein R′ is chosen from C₁-C₁₀ alkyl groups.

9. The composition according to claim 8, wherein the at least one electrophilic monomer is chosen from C₁₋₂₀ polyfluoroalkyl 2-cyanoacrylates, (C₁-C₁₀ alkyl) cyanoacrylates, and (C₁-C₄ alkoxy)(C₁-C₁₀ alkyl) cyanoacrylates.

10. The composition according to claim 9, wherein the at least one electrophilic monomer is chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, and isoamyl cyanoacrylate.

11. The composition according to claim 10, wherein the at least one electrophilic monomer is a compound of formula (V): 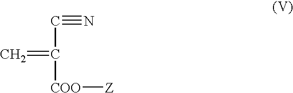
wherein Z is chosen from —(CH₂)₇—CH₃; —CH(CH₃)—(CH₂)₅—CH₃; —CH₂—CH(C₂H₅)—(CH₂)₃—CH₃; and —(CH₂)₄—CH (C₂H₅)—CH₃.

12. The composition according to claim 1, wherein the at least one electrophilic monomer is attached covalently to supports.

13. The composition according to claim 12, wherein the supports are chosen from polymers, oligomers, and dendrimers.

14. The composition according to claim 1, wherein the at least one electrophilic monomer is present in an amount ranging from 0.001% to 80% by weight, relative to the total weight of the composition.

15. The composition according to claim 14, wherein the at least one electrophilic monomer is present in an amount ranging from 0.002% to 75% by weight, relative to the total weight of the composition.

16. The composition according to claim 15, wherein the at least one electrophilic monomer is present in an amount ranging from 0.1% to 40% by weight, relative to the total weight of the composition.

17. The composition according to claim 1, wherein the cosmetically acceptable anhydrous medium is chosen from at least one of olive oil, castor oil, rapeseed oil, coconut oil, wheatgerm oil, sweet almond oil, avocado oil, macadamia oil, apricot oil, safflower oil, candlenut oil, camelina oil, tamanu oil, lemon oil, polybutene oil, isononyl isononanoate, isostearyl malate, pentaerythrityl tetraisostearate, tridecyl trimellitate, and mixtures of cyclopentasiloxane and α, ω-dihydroxylated polydimethylsiloxane.

18. The composition according to claim 1, further comprising at least one polymerization inhibitor.

19. The composition according to claim 18, wherein the at least one polymerization inhibitor is chosen from free-radical and anionic polymerization inhibitors.

20. The composition according to claim 18, wherein the at least one polymerization inhibitor is chosen from from sulphur dioxide, nitric oxide, lactone, boron trifluoride, hydroquinone and its derivatives, benzoquinone and its derivatives, catechol and its derivatives, anisole and its derivatives, hydroxyanisole, butylated hydroxyanisole, pyrogallol, 2,4-dinitrophenol, 2,4,6-trihydroxybenzene, p-methoxyphenol, hydroxybutyl-toluene, alkyl sulphates, alkyl sulphites, alkyl sulphones, alkyl sulphoxides, alkyl sulphides, mercaptans, and 3-sulpholene.

21. The composition according to claim 20, wherein the derivatives of hydroquinone are chosen from hydroquinone monoethyl ether and tert-butylhydroquinone (TBHQ).

22. The composition according to claim 20, wherein the derivative of benzoquinone is duroquinone.

23. The composition according to claim 20, wherein the derivatives of catechol are chosen from tert-butylcatechole and methoxycatechol.

24. The composition according to claim 20, wherein a derivative of anisole is methoxyanisole.

25. The composition according to claim 18, wherein the at least one polymerization inhibitor is present in an amount ranging from 10 ppm to 20% relative to the total weight of the composition.

26. The composition according to claim 1, further comprising at least one agent chosen from reducing agents; fats; plasticizers; softeners; antifoams; moisturizers; pigments; clays; mineral fillers; UV filters; mineral colloids; peptizers; solubilizers; perfumes; preservatives; anionic, cationic, nonionic, or amphoteric surfactants; fixative polymers; non-fixative polymers; polyols; proteins; vitamins; direct dyes; oxidation dyes; pearlizers; propellants; organic thickeners; and inorganic thickeners.

27. The composition according to claim 26, wherein the at least one agent is encapsulated.

28. A method of cosmetically treating hair comprising applying to the hair a cosmetic composition comprising at least one electrophilic monomer and at least one non-reducing organic acid containing from 1 to 12 carbon atoms, in a cosmetically acceptable anhydrous medium chosen from at least one of organic oils, silicones, mineral oils, plant oils, waxes, C₅-C₁₀ alkanes, acetone, methyl ethyl ketone, C₁-C₂₀ acid esters of C₁-C₈ alcohols, dimethoxyethane, diethoxyethane, C₁₀-C₃₀ fatty alcohols, C₁₀-C₃₀ fatty acids, C₁₀-C₃₀ fatty amides, and esters of C₁₀-C₃₀ fatty alcohols.

29. The method according to claim 28, further comprising applying at least one nucleophile.

30. The method according to claim 29, wherein the at least one nucleophile is chosen from molecular compounds, oligomers, dendrimers, and polymers possessing at least one nucleophilic functional group chosen from from R₂N⁻, NH₂⁻, Ph₃C⁻, R₃C⁻, PhNH⁻, pyridine, ArS⁻, R—C═C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, where Ph represents a phenyl group, Ar represents an aryl group, and R is chosen from C₁-C₁₀ alkyl groups.

31. The method according to claim 30, wherein the at least one nucleophile are chosen from hydroxyl ions.

32. The method according to claim 31, wherein the hydroxyl ions are present in water.

33. The method according to claim 28, wherein the cosmetic composition is applied to hair that has been wetted beforehand with an aqueous solution having a pH that has been adjusted by means of a base, an acid, or an acid/base mixture.

34. The method according to claim 28, wherein the hair is pre-impregnated with at least one nucleophile other than water.

35. The method according to claim 28, wherein the hair is pre-reduced before the cosmetic composition is applied.

36. The method according to claim 28, further comprising rinsing the hair after applying the cosmetic composition.

37. A multi-compartment kit comprising
a first composition comprising at least one electrophilic monomer and optionally comprising at least one polymerization inhibitor chosen from free-radical and anionic polymerization inhibitors, and
a second composition comprising at least one non-reducing organic acid containing from 1 to 12 carbon atoms in a cosmetically acceptable anhydrous medium chosen from at least one of organic oils, silicones, mineral oils, plant oils, waxes, C₅-C₁₀ alkanes, acetone, methyl ethyl ketone, C₁-C₂₀ acid esters of C₁-C₈ alcohols, dimethoxyethane, diethoxyethane, C₁₀-C₃₀ fatty alcohols, C₁₀-C₃₀ fatty acids, C₁₀-C₃₀ fatty amides, and esters of C₁₀-C₃₀ fatty alcohols.

38. A method of cosmetically treating hair, comprising
a first step comprising applying at least one non-reducing organic acid containing from 1 to 12 carbon atoms to the hair, and
a second step comprising applying at least one electrophilic monomer to the hair.

39. The method according to claim 38, wherein the at least one non-reducing organic acid is applied before the at least one electrophilic monomer is applied.
